(19) United States
(12) Patent Application Publication
(10) Pub. No.: US 2026/0085269 A1
(43) Pub. Date: Mar. 26, 2026

(54) STERILIZATION PROCESS CHALLENGE DEVICES

(71) Applicant: Noxilizer, Inc., Hanover, MD (US)

(72) Inventor: David Opie, Hanover, MD (US)

(21) Appl. No.: 19/338,980

(22) Filed: Sep. 24, 2025

Related U.S. Application Data
(60) Provisional application No. 63/698,336, filed on Sep. 24, 2024.

(51) Int. Cl. C12M 1/12; A61L 2/20; A61L 2/28; A61L 101/02; A61L 101/06; C12Q 1/22

(52) U.S. Cl. CPC C12M 37/06; A61L 2/20; A61L 2/208; A61L 2/28; C12Q 1/22; A61L 2101/02; A61L 2101/06; A61L 2103/15

(57) ABSTRACT

Sterilization process challenge devices configured to hold a biological indicator. An example includes a process sterilization device (PCD) comprising a first section of tubing defining a first internal lumen and a second section of tubing defining a second internal lumen. The first section of tubing and second section of tubing may be configured for positioning adjacent one another to form the PCD so that the first internal lumen and second internal lumen define a continuous lumen of constant inner diameter.

### CROSS REFERENCE TO RELATED APPLICATIONS

This application claims the benefit of priority of U.S. Provisional Application No. 63/698,336 filed Sep. 24, 2024, the entire disclosure of which is hereby incorporated by reference.

### TECHNOLOGY FIELD

The present application relates generally to sterilization validation and monitoring systems. More specifically, embodiments relate to process challenge devices (PCDs) for testing and validating the efficacy of gas sterilization processes in lumened devices.

### BACKGROUND

Medical device sterilization is critical for preventing healthcare-associated infections, with the goal of achieving a sterility assurance level (SAL) of 10⁻⁶ or better, meaning the probability of a non-sterile device is less than one in one million. Some medical devices present particular sterilization challenges due to their design complexity and geometry, including devices with internal lumens such as endoscopes and other hollow medical instruments.

Lumen sterilization has long been recognized as challenging for gas sterilization processes. A “lumen” is the inner hollow space of a tube, and the sterilant gas distribution along the length of the lumen may not be accurately represented by the sterilant gas concentration measured in the sterilization chamber volume during the exposure period. The efficacy of sterilization processes is typically demonstrated and monitored using biological indicators (BIs) placed at locations known to pose challenges to the sterilization process. There is an ongoing need for improved process challenge devices specifically configured for testing and validating alternative gas sterilization processes in lumened medical devices, where biological indicators can be appropriately positioned to accurately simulate the most challenging sterilization conditions encountered in actual use.

### SUMMARY

This disclosure generally provides process challenge devices (PCDs) for testing and validating the efficacy of gas sterilization processes in lumened devices.

In a first example, a process challenge device (PCD) for testing gas sterilization processes may comprise a first section of tubing defining a first internal lumen and a second section of tubing defining a second internal lumen. The first section of tubing and the second section of tubing may be configured for positioning adjacent one another to form the PCD so that the first internal lumen and the second internal lumen define a continuous lumen of constant inner diameter. At least one retention feature positioned within the continuous lumen for holding a biological indicator (BI) may be at or adjacent to an intersection of the first section of tubing and the second section of tubing.

Alternatively, or additionally, to any of the examples above, in another example, the first section of tubing and the second section of tubing may be releasably coupled to one another.

Alternatively, or additionally, to any of the examples above, in another example, the first section of tubing and the second section of tubing may be configured to interlock with one another through complementary end configurations.

Alternatively, or additionally, to any of the examples above, in another example, a first end of the first section of tubing may have a reduced outer diameter and a second end of the second section of tubing may have an increased inner diameter, such that the first end of the first section of tubing fits into the second end of the second section of tubing in either a threaded or friction fit manner.

Alternatively, or additionally, to any of the examples above, in another example, the at least one retention feature may be an interior of the second end of the second section of tubing having the increased inner diameter.

Alternatively, or additionally, to any of the examples above, in another example, the at least one retention feature may comprise one or more filaments extending across a diameter of the continuous lumen.

Alternatively, or additionally, to any of the examples above, in another example, the at least one retention feature may comprise one or more stubs or spikes extending from an inner wall of the first section of tubing and/or the second section of tubing.

Alternatively, or additionally, to any of the examples above, in another example, the at least one retention feature may comprise a coating disposed on a luminal surface of one or both of the first section of tubing and the second section of tubing.

Alternatively, or additionally, to any of the examples above, in another example, the first section of tubing may be formed of a first material for at least 80% by volume or weight thereof; and the at least one retention feature may be a portion of the first section of tubing formed of a second material different from the first material, the second material being exposed in the first internal lumen.

Alternatively, or additionally, to any of the examples above, in another example, the first section of tubing and the second section of tubing may be releasably coupled via a fitting.

Alternatively, or additionally, to any of the examples above, in another example, the at least one retention feature may be configured to pierce the BI.

In another example, a process challenge device for validating gas sterilization processes may comprise a tubular structure defining a lumen extending from a first end to a second end, at least one biological indicator (BI) positioned within the tubular structure at a location that is between 25% and 75% of a total distance from the first end to the second end, and a retention mechanism configured to maintain the at least one BI at the location during gas sterilization processing. The retention mechanism may comprise a movable element that transitions between a collapsed configuration for insertion and an expanded configuration for BI retention.

Alternatively, or additionally, to any of the examples above, in another example, the retention mechanism may comprise a coil-shaped filament.

Alternatively, or additionally, to any of the examples above, in another example, the retention mechanism may extend through the BI.

Alternatively, or additionally, to any of the examples above, in another example, the retention mechanism may comprise a shape memory material.

In another example, a method of validating a gas sterilization process may comprise providing a process challenge device comprising a tubular structure defining a lumen extending from a first end to a second end, the tubular structure having a plurality of access locations along its length, placing biological indicators at multiple positions within the lumen, including at least one biological indicator positioned at a location between 25% and 75% of a total distance from the first end to the second end, subjecting the process challenge device to a gas sterilization cycle using an alternative gas sterilization method selected from nitrogen dioxide, chlorine dioxide, or hydrogen peroxide vapor, and evaluating survival of the biological indicators at the multiple positions to determine efficacy of the gas sterilization process.

Alternatively, or additionally, to any of the examples above, in another example, the biological indicators may be placed at least at positions corresponding to 25%, 50%, and 75% of the total distance from the first end to the second end.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1A shows an illustrative tube;

FIG. 1B shows an illustrative process challenge device;

FIG. 2 shows an illustrative example, with the broken lines showing a selection thereof further highlighted in FIGS. 3-7;

FIGS. 3-7 show illustrative examples of process challenge devices;

FIGS. 8A-8C show illustrative examples of process challenge devices; and

FIG. 9 is a graph showing the calculated sterilant concentration (using a numerical model) with four different starting pressures.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION OF EXEMPLARY EMBODIMENTS

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

A wide variety of medical devices have been developed for medical use. Some devices are fully implantable (for example, orthopedic implants, stents and various electrical stimulators) to replace or repair bone, in the bloodstream or elsewhere in the body. Other devices are introduced and removed from the body in a single procedure (for example, endoscopes, catheters, and guidewires). Still other devices are used for introducing materials to or extracting material from the body (for example, syringes). Still other products are used in repairing or otherwise treating the body (for example, sutures and various staples). These devices may require sterilization.

The purpose of sterilization for medical devices is to render the device free of viable microbial life so that there is a minimized risk of infection from contact with the medical device. A sterile medical device is one where the sterilization process has demonstrably yielded a probability of a non-sterile device is less than about one in one million. This sterility level is defined as the Sterility Assurance Level (SAL).

Some medical devices may be difficult to sterilize because of complexity or geometry. Complex medical device designs may present sterilization challenges in two main ways: (1) small features that are difficult to clean and (2) surfaces that fit tightly together thus preventing sterilizing gas from accessing these blocked areas. Endoscopes are a sterilization challenge because of the design complexity and the long narrow working channels (lumens) in the device. Lumen sterilization is a recognized challenge for gas sterilization processes. A “lumen” is the inner hollow space of a tube. Lumens are a challenge to a gas sterilization process because the sterilant gas distribution along the length of the lumen is not represented by the sterilant gas concentration measured in the sterilization chamber volume during the exposure period.

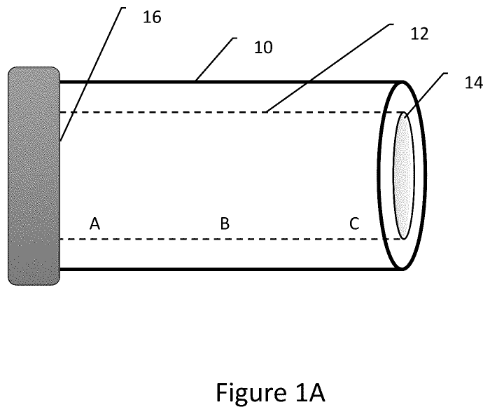

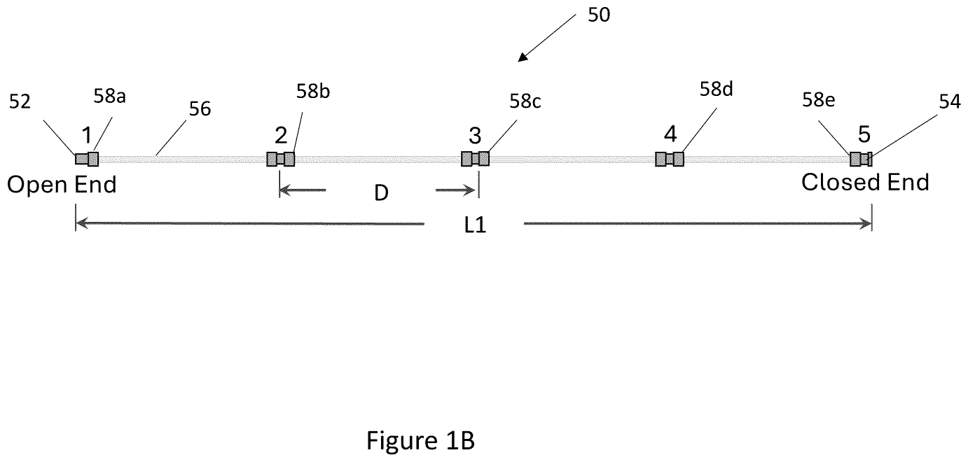

FIG. 1 is a schematic view of an illustrative lumened device 10 with a lumen 12 extending from an open end 14 to a closed end 16. However, some lumened devices may include an opening on each end thereof. With traditional sterilization methods, it is assumed that the position within the lumen 12 farthest from the lumen opening(s) 14 is the hardest-to-kill location. Thus, in FIG. 1, location A would be assumed to be the most difficult to sterilize using traditional sterilization methods, such as steam or ethylene oxide (ETO) sterilization.

With alternative gas sterilization methods, this assumption may not hold. Alternative gas sterilization methods, as used herein, refers to each of NO2, Chlorine Dioxide (ClO2), and hydrogen peroxide vapor (H202V). As the present Inventor has recognized, location A may not be the hardest to kill location in a lumen 12 with the nitrogen dioxide (NO2) sterilization process. Testing and computational models both indicate that the distance assumption simply does not hold for NO2. The mechanisms for sterilization process gases to enter a lumen are diffusion and pressure change. ETO sterilization uses relatively longer sterilant exposure phases, providing time for diffusion to achieve more homogeneous gas distribution. In contrast, alternative gas sterilization methods have shorter sterilant exposure phases, which limit the impact that diffusion can have in achieving homogeneous gas distribution within the chamber and within the load to be sterilized. Therefore, dynamical changes in pressure become the main means for alternative sterilant gases to enter lumens and result in distribution of sterilization process gases along the length of the lumens.

The distribution of the process gases, which can be humidity, sterilant gas, and diluent gases (e.g., air or nitrogen), will depend on the dynamics of the process cycle. Anticipating the hardest-to-kill location requires an understanding of the gas dynamics inherent with each alternative sterilization method. The distribution of the sterilization process gases in the lumen is directly related to the instantaneous concentration levels of all process gas constituents as the pressure is increased from the lowest to the highest pressure used in the sterilization process. Therefore, the factors influencing distribution of process gases in a lumen are the initial process pressure, the pressure change used to deliver humidity, the pressure change associated with sterilant dosing, and the pressure increase associated with the addition of diluent gases.

The efficacy of sterilization processes is typically demonstrated and monitored using biological indicators (BIs) placed at locations known to pose challenges to the sterilization process. Testing was completed using lumen process challenge devices (PCDs), with multiple lumen PCDs exposed in each of more than 40 alternative gas sterilant exposure cycles. The lumen PCDs had BIs placed at specified locations along the length of the lumen PCDs. The data shows that the location that is hardest-to-kill is not at the location farthest from the lumen opening. Computational modeling explains gas distribution within a lumen and insight as to conditions that result in the locations within the lumen that are disadvantaged during a sterilization cycle. With ETO, NO2, and ClO2, condensation of the sterilant is not a concern. However, for, H2O2V, the gas dynamics in sterilization process have an additional complication of condensation, particularly in the lumen. These observations have implications for sterilization of packaged medical devices, on process development, and lumen PCD design.

A lumen in a medical device can be open on one end and closed on the other end (as shown in FIG. 1), or the lumen can be open at both ends. For a lumen that is open on both ends, as the pressure in the sterilization chamber is increased, gas will flow into the lumen from both open ends. The midpoint in the lumen is a boundary where, as pressure in the sterilization chamber is increased, there is zero flow in the axial direction of the lumen at the midpoint. Similarly, at the closed end of a lumen that is only open on one end, there is zero flow in the axial direction of the lumen at the closed end. This means that the gas concentration distribution within a lumen that is 100 centimeters (cm) long and closed at one end will be the same as the gas distribution in each half of a lumen 200 cm long that is open at both ends. For this reason, it is sufficient to model lumens open on one end and closed on one end because the closed end represents the plane of symmetry for the calculated solution of gas distribution.

The sterilant gas that is added to the sterilization chamber reaches the interstices of a load to be sterilized in response to either pressure gradient or concentration gradient. A pressure gradient, as will occur as the gas pressure is increased in the sterilization chamber, causes sterilant gas outside of a medical device to flow into the medical device. When the pressure in sterilization chamber is stable, then a sterilant gas concentration gradient will lead to diffusion of the sterilant gas molecules from a region of higher concentration to a region of lower concentration. Fick's Second Law of Diffusion can be used to calculate the rate that diffusion causes distribution of sterilant gas in a medical device. The rate of pressure increases in the sterilization chamber, for example by adding a diluent gas, can be controlled. However, diffusion of the sterilant gas is not directly controllable.

As described herein, the efficacy of a medical device sterilization process is demonstrated and monitored with BIs that are placed at locations that are known to pose a challenge to the sterilization process. Such locations that can be challenged with BIs are locations within a packaged medical device. When a lumen is part of the medical device to be sterilized, it is assumed that the location farthest from the lumen opening or openings is the hardest-to-kill location.

To simulate the challenge of sterilizing a lumen, a Process Challenge Device (PCD) can be used. These PCDs may have a holder for the BI which places the BI at the location farthest from the lumen opening. One such PCD consists of a stainless-steel tubing that is 4550 millimeters (mm) long and with a 2 mm internal diameter (ID). This PCD has a sealable BI holder (0.85 milliliter (ml) volume) at one end so that the gas that reaches the BI must traverse the entire length of the lumen.

To simulate the lumens found in endoscopes, there are specific process challenge devices that are meant to simulate the challenges associated medical devices with long narrow lumens. For example, a type-test PCD that follows the requirements of EN 1422:2009, Annex F (Biological performance type test for Type B ethylene oxide sterilizers) can be used to demonstrate sterilization of lumened devices with lumens of length and diameter represented by the type-test PCD. This process challenge device consists of stainless-steel or polymeric tubing with a gastight capsule for holding the biological indicator (the BI consists of a carrier material on which the spores are inoculated). The gastight capsule can be opened to place and retrieve the BI. These PCDs can be purchased from multiple suppliers (e.g., some may be commercially available from GKE, Waldems, Germany, etc.).

As will be shown herein, this type of PCD is not appropriate for alternative gas sterilization processes, like nitrogen dioxide, hydrogen peroxide, and chlorine dioxide sterilization processes. One would assume the hardest-to-kill location to correspond with the location of lowest sterilant concentration. The position farthest from the lumen opening(s) may not be the location of lowest sterilant concentration, and therefore may not be the hardest-to-kill location. Additionally, the additional volume associated with the BI holder changes the distribution of gas in the lumen. Therefore, an appropriate PCD configuration is needed for alternative sterilization processes.

Testing was completed using lumen process challenge devices (PCDs), with multiple lumen PCDs exposed in each of more than 40 sterilant exposure cycles. The lumen PCDs had BIs placed at specified locations along the length of the lumen PCDs. Testing shows that the hardest to-kill-location is not at the location farthest from the lumen opening. FIG. 1B is a schematic view of an illustrative PCD 50 that was used for testing. The PCD 50 extends from a first open end 52 to a second closed end 54. The PCD 50 has a length L1 extending from the first open end 52 to the second closed end 54 with a lumen extending along the length L1. In the illustrated embodiment, the length L1 is 150 cm. However, the length L1 may be less than 150 cm or greater than 150 cm, as desired. The PCD 50 was a polymer tube 56 defining a lumen with stainless steel fittings 58a-e evenly spaced by a distance D so that the lumens could be opened at the fitting locations for BI placement, and after which the fittings sealable closed. BIs were placed at five evenly spaced positions. Position 1 was at the opening of the lumen and position 5 was at the closed end. In the illustrated example, position 2 was 37.5 cm from the opening, position 3 was 75 cm from the opening, and position 4 was 112.5 cm from the opening. The lumen internal diameter (ID) was 3.6 mm. The stainless-steel fittings 58a-e had an ID that matched the polymer lumen ID. The BIs were 3 mm diameter discs with small gauge stainless steel wire to keep the BIs from moving during placement, handling, testing, and/or recovery. The test NO2 sterilant exposure conditions were 80% RH, 6-minute dwell, and the sterilant concentration was varied from 2 mg/L to 10 mg/L. This cycle was intentionally chosen to be only marginally successful for the device.

The results of the testing show that for all sterilant exposure cycles, the BIs were sterilized at positions 1 and 5. Table 1 below shows the number of positive BIs over the number of BIs tested for the sterilant concentration shown. For example, 0/2 indicates zero out of two BIs tested positive. The results indicate that a lumen PCD requires placement of BIs at positions other than the point farthest from the lumen opening. For example, a lumen PCD could have one or more fittings so that BIs can be placed at positions between the opening and the closed end of the lumen.

TABLE 1
Number of Positive BIs at Each Location

| | Position | | | | |
|---|---|---|---|---|---|
| NO2 | 1 | | | | |
| Concentration | (lumen | | | | 5 (closed |
| (mg/L) | opening) | 2 | 3 | 4 | end) |
| 2 | 0/2 | 2/2 | 2/2 | 0/2 | 0/2 |
| 3 | 0/4 | 3/4 | 4/4 | 1/4 | 0/4 |
| 4 | 0/2 | 0/2 | 2/2 | 0/2 | 0/2 |
| 5 | 0/5 | 3/5 | 3/5 | 1/5 | 0/5 |
| 6 | 0/4 | 1/4 | 0/4 | 0/4 | 0/4 |
| 8 | 0/2 | 0/2 | 0/2 | 0/2 | 0/2 |
| 10 | 0/2 | 0/2 | 0/2 | 0/2 | 0/2 |
| All | 0/21 | 9/21 | 11/21 | 2/21 | 0/21 |
| | 0% | 43% | 52% | 10% | 0% |

In examples, the fittings used for BI placement should not include a diameter larger than the length of the lumen represented by the PCD. An enlargement of the diameter permits gas mixing before the gas can move farther into the lumen. Such mixing alters the distribution of gas along the length of the lumen that is distal to the section with a larger diameter.

Additionally, the PCD may include features in the fitting or lumens for BI retention that keep the BI at the desired test location and that prevent the BI from sliding along the length of the lumen during handling, exposure, and/or recovery. Any such feature or features should not significantly block gas flow into and/or out of the lumen. Such features might be one or more protrusions in the fittings or the lumen wall near the fittings that project into the volume within the lumen. This feature could be one or more small rods protruding from the fitting or lumen wall that would facilitate retention of the BI at the desired test location. Alternatively, one or more thin wires or filaments could be used for BI retention at the desired test location. These wires or filaments could be a coil or twisted member that block the BI from moving along the length of the lumen. Alternatively, the one or more wires or filaments could transect across the diameter of the lumen, as a secant line or as a bisecting line.

With a lumen PCD having openings at both ends, all the foregoing applies. The fittings for BI placement could be at selected positions along the entire length of the lumen PCD.

An equivalent volume can be used in place of a length of lumen more distant from the opening than a desired test location. Calculations of the gas flow into the lumens will demonstrate that a volume of any shape can be used if this equivalent volume equals the volume of the lumen that is replaced by the equivalent volume. For example, testing of this principle used a modified lumen PCD in which the tubing more distal from third fitting 58c (e.g., position 3) was replaced with tubing having an equivalent volume. The equivalent volume had large diameter section that was more than 10 mm in internal diameter and of sufficient length to equal a same volume as 75 cm of the smaller inner diameter (e.g., 3.6 mm) lumen PCD tubing 56.

For this test, the lumen opening is at position 1. The lumen is closed at a point farthest from the lumen opening but is closed with an enlarged diameter at position 4. Said differently, the diameter of the lumen increases distal to position 3 and a distance from position 3 to the closed end (now position 4) is less than the distance in the example PCD of FIG. 1B. BIs were placed at position 1, position 2, position 3 and 4 (in this large diameter volume). The BI in position 3 (e.g., in the smaller diameter lumen proximal to the larger diameter portion) survived cycles and the BIs in the large diameter volume were sterile. The BIs at positions 1 and 2 were sterile. The test NO2 sterilant exposure conditions with the equivalent volume portion were 80% RH, 6-minute dwell and the sterilant concentration was varied from 3 mg/L to 6 mg/L. Table 2 below shows the number of positive BIs over the number of BIs tested for the sterilant concentration shown. For example, 0/1 indicates zero out of one BIs tested positive.

TABLE 2
Number of Positive BIs at Each Location

| | |
|---|---|
| NO2 | BI Position |

| Concentration | | | | 4 (Large |
|---|---|---|---|---|
| (mg/L) | 1 | 2 | 3 | Volume) |
| 3 | 0/1 | 0/1 | 1/1 | 0/1 |
| 5 | 0/1 | 0/1 | 1/1 | 0/1 |
| 6 | 0/1 | 0/1 | 0/1 | 0/1 |

This test demonstrates that a holder of BIs in a lumen that has a diameter that is enlarged to hold the BI is not a challenge of the hardest-to-kill position in the lumen and is not an appropriate lumen challenge for the sterilization process.

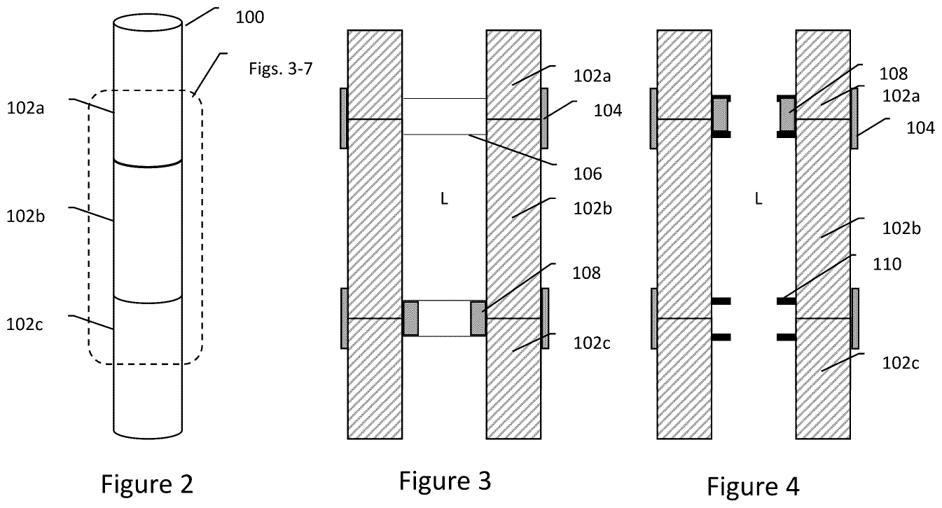

FIG. 2 shows an illustrative example process challenge device 100, with the broken lines showing a selection thereof further highlighted in FIGS. 3-7. The process challenge device 100 includes a plurality of sections 102a, 102b, 102c fitted together. Each section 102a-c is tubular, and may be of any suitable length, for example, from 3 cm to 10 cm, or longer or shorter, with an inner diameter in the range of about 3 to about 30 French, or larger or smaller. The lumen thus defined may have a circular, oval, polygonal, or other internal shape, as desired. The sections 102a-c may slide together, forming a friction fit, or may be screwed together. In some cases, fittings may be used to couple the sections 102a-c together. In some examples, sidewalls of one or more of the sections 102a-c may include one or more through holes so that one or more threads can be strung therethrough to secure the entire length of the assembly together. However, this is not required.

FIGS. 3-7 show illustrative examples of providing access locations and/or securement mechanisms for positioning BIs in a PCD 100. A challenge to solve is how to create locations in which BI can be accessed and also secured in place without causing the internal lumen, L, to have undesired irregularities that interfere with the sterilant gas passing therethrough. Further, in some cases, the BIs may be formed from inoculated quartz filter media which are lightweight and may be easily displaced by pressure changes or the like. Multiple sections 102a-c configured to be selectively coupled and uncoupled may provide multiple access locations. Process validation may typically allow the BI to interfere, to some extent, with sterilant gas movement, though such interference is to be minimized.

In FIG. 3, several sections 102a, 102b, 102c are shown. Exterior fittings 104 may be used to secure sections in place adjacent one another. Threads or filaments 106 may cross the lumen, forming chords that hold a BI 108 in position so it will not slide up or down the inner lumen as pressure changes occur and gas passes into the lumen. The threads are positioned near, but spaced from, the ends of each section 102a, 102b, 102c, creating spacing there between to match the size of the BI 108.

In FIG. 4, threads 106 are replaced with short stubs or spikes 110, again, to hold the BI 108 in position. Each stub or spike 110 is again positioned near but spaced from the end of each section 102a, 102b, 102c, to hold the BI 108. Any suitable material may be used for the threads 106, stubs or spikes 110 and/or sections 102a, 102b, 102c.

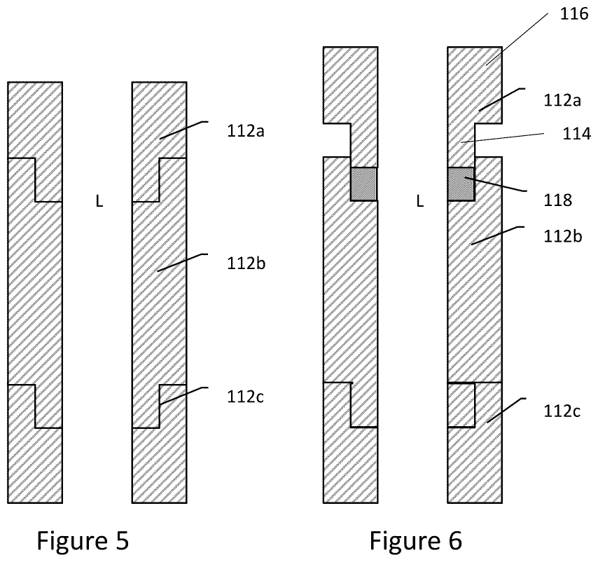

FIGS. 5-6 show another example, with interlocking sections 112a, 112b, 112c, having first ends 114 of narrower outer diameter, and second ends 116 of greater inner diameter, which can interlock as shown. In some configurations, one or more intermediate sections, such as, but not limited to, intermediate section 112b, may have a narrower outer diameter at each end thereof. The narrower outer diameter ends 114 may be arranged or offset such that a narrower outer diameter end 114 mates with an adjacent narrower outer diameter end 114. In some examples, the ends 114, 116 may include releasable interlocking mechanisms such as, but not limited to, snap fits, friction fits, etc., configured to be repeatedly coupled and uncoupled. This design presents a consistent inner lumen size, avoiding interference with the passage of sterilant gas. As shown in FIG. 6, the BI 118 can be positioned between sections when the ends of adjacent sections 112a, 112b, 112c are spaced from one another. The result is a consistent inner diameter, and the BI 118 does not interfere with passage of sterilant gas. When the interlocking sections 112a, 112b, 112c are fully interlocked, the internal lumen defined is consistent. Said differently, inner wall defining the lumen L may have a uniform diameter and may be substantially continuous. When a BI 118 is to be positioned, the interlocking sections 112a, 112b, 112c are only partly interlocked, leaving gaps in which the BI 118 is positioned. A cylindrical BI may be used, if desired, to maintain constant inner diameter, if desired, although this is not required.

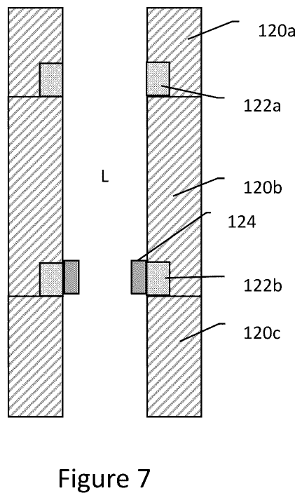

FIG. 7 shows another example. Here, the material of the 120a, 120b, 120c may be hard or slippery (lubricious) if desired, which may match the material used in a particular device to be sterilized. Small segments, 122a, 122b of at least some sections (such as, but not limited to, sections 120a, 120b) are made of, or coated with, a second material that is more sticky than the remainder of the section, thus holding the BI 124 at a desired position. An example includes sections 120a, 120b that are formed primarily of a first material (at least 80% by volume or weight), where small segments (portions 122a, 122b) within sections 120a, 120b are formed of a second, different material and are exposed within the internal lumen L to serve as a mechanism for holding the BI 124.

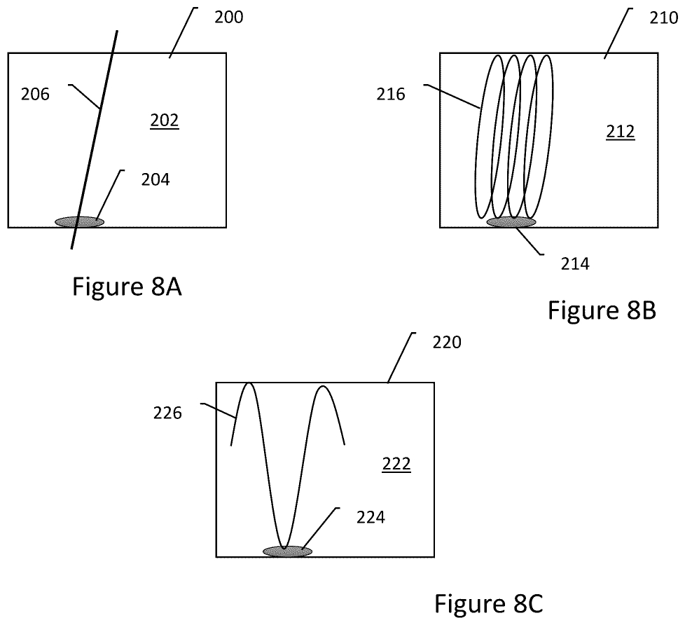

FIGS. 8A-8C illustrate additional devices and systems for securing a BI within a lumen of a PCD. FIG. 8A is a schematic partial cross-sectional view of an illustrative PCD 200 defining a lumen 202. The BI 204 may be positioned at the desired location within the lumen 202. This may be done using a segmented PCD 200 (e.g., having two or more tubular members releasably coupled to one another). The segmented PCD 200 may be releasably coupled using any of the techniques described herein. However, it is not required for the PCD 200 to be segmented. Once the BI 204 is in the desired position a needle 206, or other piercing member, may be pierced through the PCD 200 and the BI 204 to hold the BI 204 in a desired location. The needle 206 may be formed from a material (e.g., metals, plastics, composites, or the like) configured to pierce a plastic tube forming the PCD 200. It is contemplated that the needle 206 may have a cross-sectional dimension (taken orthogonal to a longitudinal axis thereof) that is sufficiently small to minimize disruptions to process gas flow within the lumen 202.

FIG. 8B is a schematic partial cross-sectional view of another illustrative PCD 210 defining a lumen 212. The BI 214 may be positioned at the desired location within the lumen 212. This may be done using a segmented PCD 210 (e.g., having two or more tubular members releasably coupled to one another). The segmented PCD 210 may be releasably coupled using any of the techniques described herein. However, it is not required for the PCD 210 to be segmented. Once the BI 214 is in the desired position a coil, spring or other filament 216 may be deployed within the lumen 212 to entangle the BI 214 to hold the BI 214 in a desired location. In some examples, the filament 216 may be configured to position the BI 214 between the filament 216 and the inner wall of the PCD 210. Alternatively, or additionally, the BI 214 may be entangled within windings of the filament 216. While the illustrative filament 216 is shown as having uniformly sized and spaced windings, the filament 216 may have eccentrically sized and/or shaped windings, as desired. It is further contemplated that the filament 216 may include any number of windings desired. In some cases, the filament 216 may have less than one complete winding. In some embodiments, the filament 216 may be movable between a collapsed configuration having a reduced diameter configuration to facilitate insertion into the lumen 212 and an expanded configuration configured to minimize an amount of the filament 216 extending across or within the lumen 212. The filament 216 may be formed from a material configured exert a biasing force against the BI 214 or to retain an expanded shape to entangle the BI. In some examples, the filament 216 may be formed from shape memory alloys or polymers. For example, the filament 216 may be biased into a reduced diameter configuration at a reduced temperature (e.g., refrigeration) and resume an expanded configuration when heated.

FIG. 8C is a schematic partial cross-sectional view of another illustrative PCD 220 defining a lumen 222. The BI 224 may be positioned at the desired location within the lumen 222. This may be done using a segmented PCD 220 (e.g., having two or more tubular members releasably coupled to one another). The segmented PCD 220 may be releasably coupled using any of the techniques described herein. However, it is not required for the PCD 220 to be segmented. In some examples, the BI 224 may be pierced with a filament 226 prior to pushing the BI 224 into the lumen 222. For example, the filament 226 may be threaded through fabric of the BI 224. The filament 226 may have a coil or spring-like shape configured to conform to the luminal surface of the PCD 220 such that the BI 224 is held against the luminal surface of the PCD 220. While the illustrative filament 226 is shown as having uniformly sized and spaced windings, the filament 226 may have eccentrically sized and/or shaped windings, as desired. It is further contemplated that the filament 226 may include any number of windings desired. In some cases, the filament 226 may have less than one complete winding. In some embodiments, the filament 226 may be movable between a collapsed configuration having a reduced diameter configuration to facilitate insertion into the lumen 222 and an expanded configuration configured to minimize an amount of the filament 226 extending across or within the lumen 222. The filament 226 may be formed from a material configured to exert a biasing force against the luminal wall of the PCD 220.

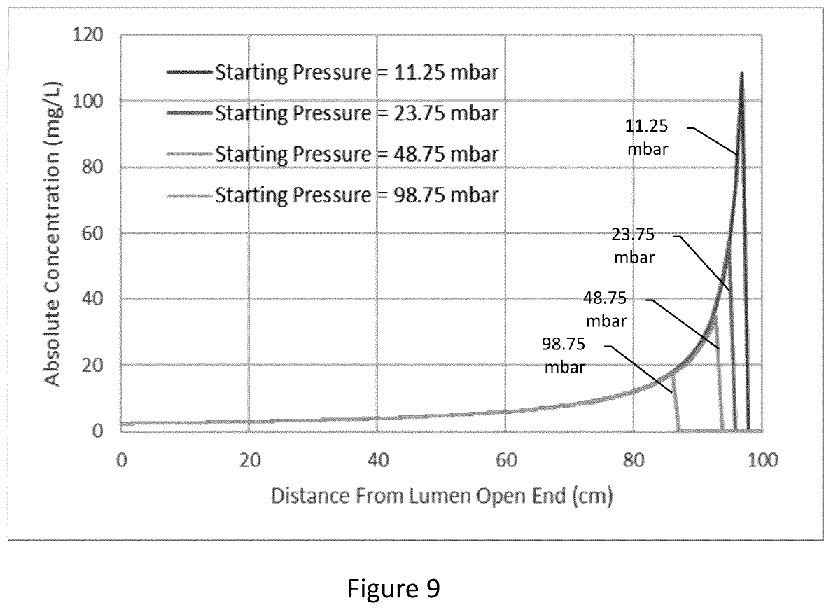

FIG. 9 is a graph showing the calculated sterilant concentration (using a numerical model) with four different starting pressures. For the graph of FIG. 9 only sterilant and air were used in the calculation. The amount of sterilant added to the sterilization chamber for each curve shown in FIG. 9 is the same (2.3 milligrams per liter (mg/L)) and only the starting pressure of the sterilant addition step is changed. The results in FIG. 9 illustrate that more sterilant enters the lumen than is in the sterilization chamber. Additionally, reducing the sterilant addition starting pressure reduces the total sterilant in the lumen.

As can be seen in FIG. 9, there is a portion of lumen, farthest from the opening that is without sterilant. The length of this sterilant-free portion increases with increasing starting pressure. The extent of this dead space for the four scenarios in FIG. 9 shows that the role of diffusion is more important for the higher starting pressure for sterilant addition.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A process challenge device (PCD) for testing gas sterilization processes, comprising:
a first section of tubing defining a first internal lumen; and
a second section of tubing defining a second internal lumen;
wherein the first section of tubing and the second section of tubing are configured for positioning adjacent one another to form the PCD so that the first internal lumen and the second internal lumen define a continuous lumen of constant inner diameter; and
at least one retention feature positioned within the continuous lumen for holding a biological indicator (BI) at or adjacent to an intersection of the first section of tubing and the second section of tubing.

2. The process challenge device of claim 1, wherein the first section of tubing and the second section of tubing are releasably coupled to one another.

3. The process challenge device of claim 2, wherein the first section of tubing and the second section of tubing are configured to interlock with one another through complementary end configurations.

4. The process challenge device of claim 3, wherein a first end of the first section of tubing has a reduced outer diameter and a second end of the second section of tubing has an increased inner diameter, such that the first end of the first section of tubing fits into the second end of the second section of tubing in either a threaded or friction fit manner.

5. The process challenge device of claim 4, wherein the at least one retention feature is an interior of the second end of the second section of tubing having the increased inner diameter.

6. The process challenge device of claim 1, wherein the at least one retention feature comprises one or more filaments extending across a diameter of the continuous lumen.

7. The process challenge device of claim 1, wherein the at least one retention feature comprises one or more stubs or spikes extending from an inner wall of the first section of tubing and/or the second section of tubing.

8. The process challenge device of claim 1, wherein the at least one retention feature comprises a coating disposed on a luminal surface of one or both of the first section of tubing and the second section of tubing.

9. The process challenge device of claim 1, wherein the first section of tubing is formed of a first material for at least 80% by volume or weight thereof; and
the at least one retention feature is a portion of the first section of tubing formed of a second material different from the first material, the second material being exposed in the first internal lumen.

10. The process challenge device of claim 1, wherein the first section of tubing and the second section of tubing are releasably coupled via a fitting.

11. The process challenge device of claim 1, wherein the at least one retention feature is configured to pierce the BI.

12. A process challenge device for validating gas sterilization processes, comprising:
a tubular structure defining a lumen extending from a first end to a second end;
at least one biological indicator (BI) positioned within the tubular structure at a location that is between 25% and 75% of a total distance from the first end to the second end; and
a retention mechanism configured to maintain the at least one BI at the location during gas sterilization processing, wherein the retention mechanism comprises a movable element that transitions between a collapsed configuration for insertion and an expanded configuration for BI retention.

13. The process challenge device of claim 12, wherein the retention mechanism comprises a coil-shaped filament.

14. The process challenge device of claim 12, wherein the retention mechanism extends through the BI.

15. The process challenge device of claim 12, wherein the retention mechanism comprises a shape memory material.

16. A method of validating a gas sterilization process, comprising: providing a process challenge device comprising a tubular structure defining a lumen extending from a first end to a second end, the tubular structure having a plurality of access locations along its length;
placing biological indicators at multiple positions within the lumen, including at least one biological indicator positioned at a location between 25% and 75% of a total distance from the first end to the second end;
subjecting the process challenge device to a gas sterilization cycle using an alternative gas sterilization method selected from nitrogen dioxide, chlorine dioxide, or hydrogen peroxide vapor; and
evaluating survival of the biological indicators at the multiple positions to determine efficacy of the gas sterilization process.

17. The method of claim 16, wherein the biological indicators are placed at least at positions corresponding to 25%, 50%, and 75% of the total distance from the first end to the second end.
